(19) United States
(12) Patent Application Publication
(10) Pub. No.: US 2011/0054433 A1
(43) Pub. Date: Mar. 3, 2011

(54) WEB FOR RETENTION OF INTERNAL BODILY SECRETIONS

(75) Inventor: Bernard Chaffringeon, Granges-Paccot (CH)

(73) Assignee: BTampon IP, Capellen (LU)

(21) Appl. No.: 12/921,963

(22) PCT Filed: Mar. 11, 2009
(86) PCT No.: PCT/EP09/52867

§ 371 Date: Oct. 25, 2010

Related U.S. Application Data
(60) Provisional application No. 61/064,565, filed on Mar. 12, 2008.
(60) Provisional application No. 61/064,982, filed on Apr. 7, 2008.

(51) Int. Cl. A61F 13/15; A61B 19/02

(52) U.S. Cl. 604/385.17; 206/440

(57) ABSTRACT

A flexible retention web designed to be introduced into a vaginal cavity, said web being provided with means of removal and being made of a non-expandable or minimally expandable atraumatic material, the dimensions of which are designed in such a way as to be able to retain and/or slow down bodily discharges of small quantity.

The present invention relates to the field of devices for absorbing natural bodily discharges, for example menstrual flows and cervical mucus.

Many sanitary articles exist, especially for absorbing the menstrual flows, for example catamenial and/or sanitary tampons made of absorbent material in the form of sheets and/or bands that are folded and/or rolled up, using techniques well known to persons skilled in the art, in order to form bodies that are able to dilate once they have been placed, with or without applicators, into the vaginas of the users.

This type of device is presently used regardless of the volume of secretions to be absorbed and is generally designed in such a way as to be able to retain a moderate or even substantial flow, in order that it is suitable for the greatest possible number of persons.

However, users who produce bodily discharges of small quantity are faced with the fact that the tampon that has been fitted is not appropriate to the volume of their secretions. The contact of the rough and more or less dilated tampon against the dry wall of the vagina results in pain, local irritation and thus causes trauma to the person using it. This sensation of discomfort is all the more pronounced when the tampon is being removed. Moreover, independently of the question of user comfort, it should be noted that the use of such large amounts of material for absorbing small volumes of discharges runs counter to the generally accepted environmental policy of reducing the amounts of waste material.

To improve tampons of this kind, document U.S. Pat. No. 6,177,608 discloses a tampon comprising an absorbent core and a permeable cover which is disposed around the core and which is provided with strips which form funnels that can be spread out from the core in such a way as to obstruct a free space formed between the outer surface of the tampon and the wall of the vagina. This makes it possible in particular for the fluid received by the funnels to converge in the direction of the absorbent core.

Moreover, document WO 03/051257 provides a flexible tampon comprising a substantially flat absorbent core arranged between two protective permeable layers. Such a tampon is intended to be used in place of a traditional tampon and is distinguished by the fact that it can be folded around the user's finger so as to be introduced gently into the vaginal cavity.

However, this principle for the design of a tampon was anticipated by many years by the document U.S. Pat. No. 1,884,089 which proposes the production of a sanitary article in the form of a disc which is made of a highly compressed absorbent material in order to absorb standard volumes or even copious volumes of bodily discharges. This disc is folded around the finger and introduced into the vaginal cavity with the finger, and a string forming a means of removal is securely attached to the centre of the disc. However, the zone of attachment of the string to the disc constitutes a hard spot that may damage the wall of the vagina during the use or during the removal of the disc. The same applies to document WO 03/051257 where the attachment of the string to the tampon likewise creates a hard spot.

The object of the present invention is to solve all or some of the abovementioned problems and, to this end, the invention proposes a novel type of sanitary article, intended more particularly for users who produce small volumes of bodily secretions.

To this end, the present invention relates to a flexible retention web designed to be introduced into a vaginal cavity, said web being provided with means of removal and being made of a non-expandable or minimally expandable atraumatic material, the dimensions of which are designed in such a way as to be able to retain and/or slow down bodily discharges of small quantity.

Within the meaning of the present invention, it should be understood that the expression “small quantity” means that the web is solely intended to retain bodily discharges in volumes much lower than those normally collected by traditional tampons, especially during menstruation. To this end, the web is designed to have no excess thickness of unnecessary material.

Moreover, within the meaning of the present invention, it should be noted that the expression “non-expandable or minimally expandable” is not to be interpreted too narrowly and it means simply that the capacity of expansion or of dilation of the web is not like that of a traditional tampon in which the core is designed to dilate in order to be able to absorb a maximum quantity of fluid.

Such a web is therefore particularly designed to afford exceptional comfort to a user who produces small amounts of bodily discharge, for example as a result of regular use of a contraceptive, or whose menstrual flow is limited, particularly at the end of the cycle.

A web according to the invention can be introduced into the vaginal cavity with the aid of a finger or a pushing tool.

Advantageously, such a web is made of a material chosen from the group comprising in particular a non-woven textile, polyvinyl acetate, cotton, an organic material, or a plant material. This material can advantageously be biodegradable.

Advantageously too, the web can have a substantially rectangular, square, oval or circular shape, for example.

Moreover, the web can be partially or completely covered by at least one agent that has healing, lubricating, anticoagulant or antioxidant properties, for example.

According to one feature of the invention, the web is continued by at least two strings constituting the means of removal.

According to a preferred variant of the invention, the means of removal are formed integrally with said web. Consequently, the web and the means of removal will be produced in one piece from the same atraumatic material. Such an arrangement is particularly advantageous since it is not necessary to fix the strings in the web by welding, adhesive bonding or another traditionally used technique. There is therefore no hard spot likely to come into contact with the wall of the vagina.

The web preferably comprises two flaps, each of them continued by a string constituting a means of removal.

According to one embodiment of the invention, the web comprises two flaps which are folded onto each other and attached to each other by connecting means in such a way as to form two lateral connections. Indeed, even though the web can be made “flat”, that is to say without the flaps being connected to each other, it is preferable to provide these lateral connections so as to delimit a cavity designed to accommodate a finger or a pushing tool. It must be understood, however, that it is not necessary for the web to be given the appearance of a pouch hermetically sealed along its sides since, on the one hand, the intention is not especially to incorporate a separate absorbent element in the web thus assembled and, on the other hand, it aims simply to ensure the function of lateral guiding of the finger or pushing tool at the time of insertion of the web.

It must be understood that these flaps will be advantageously symmetrical, in such a way as to have an equivalent surface area of retention, although they will alternatively be able to have different dimensions.

The web preferably has two lateral edges, and each of the two lateral connections is arranged set back from the lateral edge corresponding to it. In this way, it is possible to avoid creating a lateral ridge of double thickness that could form a hard spot at each of the two lateral connections. The flaps are joined to each other, but each lateral edge remains free to deform independently of the other lateral edge. Consequently, each lateral edge of small thickness is in contact with the wall of the vagina in an atraumatic manner, while the two lateral connections remain spaced apart from the wall of the vagina.

According to a particular embodiment of the invention, the connecting means are formed with the aid of at least two separate, loosely sewn strings. This variant is also very advantageous, because the strings can, where appropriate, serve as the means of removal and at the same time as the connecting means, without actually creating a hard spot. It must be understood that sewing these strings loosely allows each flap to move relative to the other flap. Thus, each flap will be able to absorb the movements of oscillation of the vaginal cavity independently of the other flap, the result of which will be to avoid the creation of micro-friction that generates local irritation of the wall of the vagina.

According to another variant embodiment, the connecting means are means of connection by buttoning. It will be advantageous to form the connecting means in this case in combination with means of removal formed in one piece with the web. The user will thus be in possession of a folded web forming a sachet in such a way as to make insertion of the web easier, without any hard spot being generated.

Preferably, each flap comprises two lateral edges in each of which the means of connection by buttoning are arranged in the form of at least one slit and/or at least one button, in such a way as to be able to cooperate respectively with at least one complementary button and/or at least one complementary slit of the flap facing it. Preferably too, at least one button has substantially the shape of a T.

According to another embodiment of the invention, the first flap of the web is longer than the second flap.

Preferably, the first flap comprises at least one opening for the passage of the string of the second flap after the latter has been turned over onto the first flap by being folded about an axis. As the flaps are asymmetrical, it is advantageous, with a view to permitting easy removal of the web, that the length of the string of the second flap is longer than that of the first flap, so as to compensate for the length difference between the two flaps.

Preferably too, the opening is made in the form of a cut substantially shaped as a T or as an inverted T.

Advantageously, the string continuing the second flap has a widened end at the zone of connection to the second flap. Thus, it is then possible to form a web in the shape of a sachet once the free end of the string has passed through the opening and once its widened end has come into abutment against the first flap.

Advantageously too, a slit is formed in proximity to the axis and is substantially parallel to the axis. This makes it possible to create a receiving area for the end of a pushing tool engaged in the web.

According to another feature, a web according to the invention comprises retaining means for limiting the outward migration of said web once it has been introduced into the vaginal cavity.

Advantageously, the retaining means comprise at least one element forming a harpoon and obtained by making a V-shaped cut in said web. In addition to this or alternatively, the retaining means can comprise at least one notch formed in the edge of the web, this notch preferably being of substantially triangular shape.

However, it must be understood that the retaining means can be obtained from any type of cut allowing the expected function to be achieved, for example an L-shaped cut that also promotes the passage of the fluid inside the web.

Moreover, it must be understood that the slits/buttons formed in the web and defined above can also serve as fastening points that limit the risk of migration of the web.

Advantageously too, and in such a way as to facilitate insertion of the web and to avoid undesired irritation, these cuts can be produced in such a way that the cross section of entry of the folded web is reduced in width and is free of any sharp point or angle.

According to another variant embodiment of the invention, the web comprises means for channelling the bodily discharges. These means will thus act as baffles which increase the surface area of contact between the web and the fluid that is to be retained, doing so in all directions.

Advantageously, the channelling means are obtained by at least one cut formed in said web, this cut preferably being in the shape of an inverted T.

According to a particularly advantageous embodiment of the invention, the web, after folding, has at least two flaps, each of them provided with at least one pocket in such a way as to increase the capacity for retention of the bodily discharges.

Each of the pockets is preferably formed by folding a part of the flap on itself and is attached to the latter by connecting means. As before, these connecting means can be in the form of loosely sewn strings at the edge, but they are preferably means of connection by buttoning, which are designed in such a way as to allow the two flaps to be attached to each other while at the same time allowing each pocket to be attached to its corresponding flap.

According to one feature of the invention, the web incorporates at least one fluid barrier formed with the aid of a hydrophobic material, for example petroleum jelly or beeswax.

The present invention also relates to a method of production of a web according to the invention by stamping a thickness of appropriate atraumatic material on a suitable die.

This method of production preferably comprises the following steps:
cutting out at least one slit and at least one button in the web,
folding the web about an axis, then introducing each button into at least one corresponding slit with the aid of a jet of air or a pushing element.

The present invention also relates to a method of production of a web according to the invention by cutting a thickness of appropriate atraumatic material in a predetermined manner with the aid of a cutting tool or a laser.

Preferably, this method of production comprises the following steps:
cutting out at least one slit and at least one button in the web,
folding the web about an axis, then introducing each button into at least one corresponding slit with the aid of a jet of air or a pushing element.

The present invention also relates to a method of production of a web by stamping a thickness of appropriate atraumatic material on a suitable die in such a way as to form a web that is continued at each end by a string.

Advantageously, this method of production comprises the following steps:
cutting out at least one opening in the web,
then passing through the opening the end of the string furthest from said opening, after folding the web onto itself about an axis.

Advantageously too, this method of production comprises the following steps:
cutting out at least one slit in the web in proximity to the axis,
inserting an end of a pushing tool through the opening until this end lodges in the slit.

The present invention also relates to a method of production of a web by cutting a thickness of appropriate atraumatic material in a predetermined manner with the aid of a cutting tool or a laser in such a way as to form a web that is continued at each end by a string.

Advantageously, this method of production comprises the following steps:
cutting out at least one opening in the web,
then passing through the opening the end of the string furthest from said opening, after folding the web onto itself about an axis.

Advantageously too, this method of production comprises the following steps:
cutting out at least one slit in the web in proximity to the axis,
inserting an end of a pushing tool through the opening until this end lodges in the slit.

The present invention also relates to a dispenser for dispensing a plurality of webs according to the invention, said dispenser being of the roller type and the webs being folded in succession between two flexible protective bands.

Advantageously, the bands each comprise a zone of weakness between two successive webs. This allows the user to more easily tear off the sections of the protective bands covering a web.

The invention will be better understood from the detailed description given below and by reference to the attached drawing, in which:

FIG. 1 is a schematic plan view of a web according to the invention;

FIG. 2 is a view similar to that of FIG. 1, once the web has been folded;

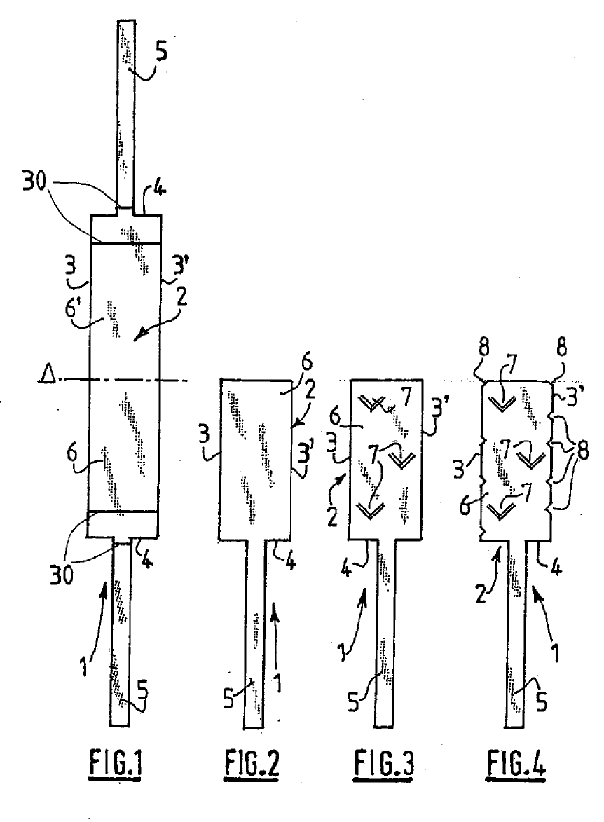

FIGS. 3 and 4 are views similar to that of FIG. 2, the web being provided with retaining means;

FIG. 5 is a view similar to that of FIG. 1, the web being provided with channelling means and with means of connection by buttoning;

FIG. 6 is a view of the web shown in FIG. 5 once it has been folded and assembled;

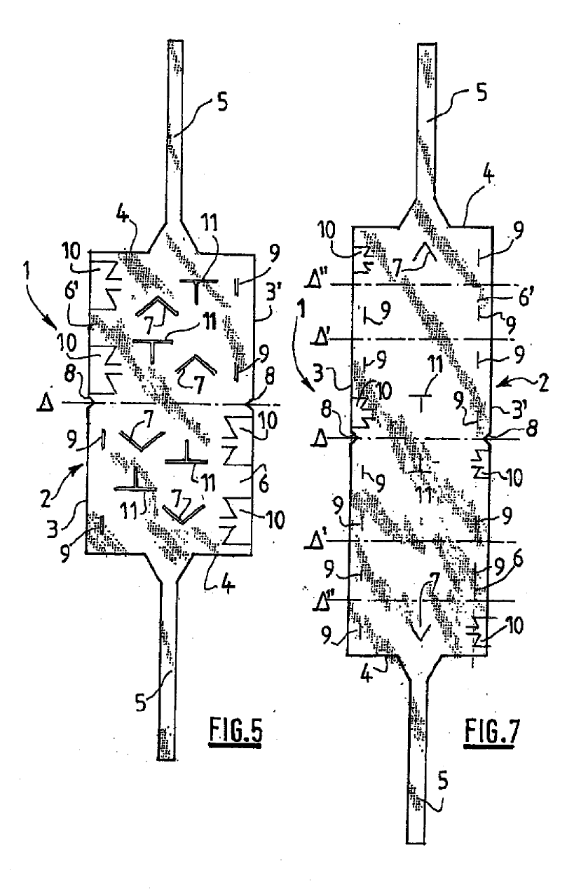

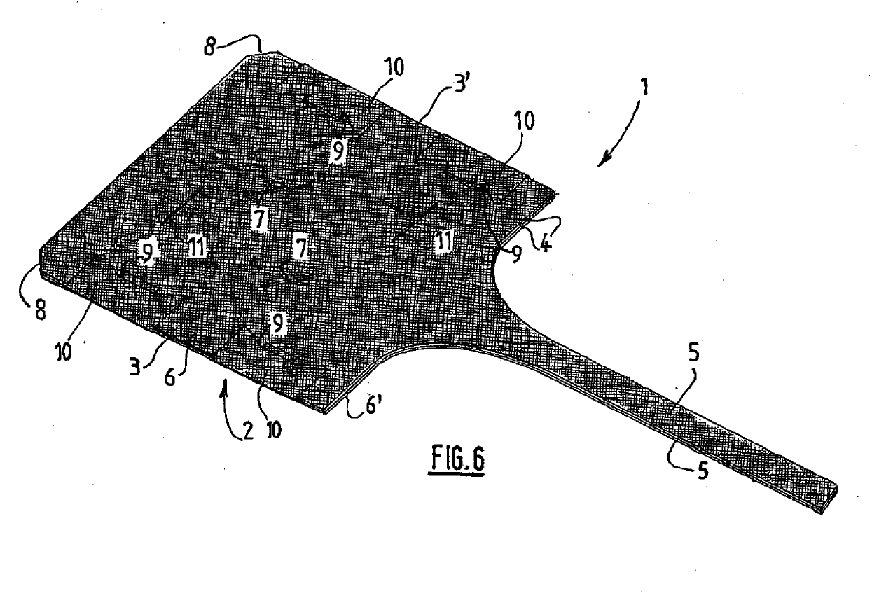

FIG. 7 is a view similar to that of FIG. 5, the web being designed to have two pockets;

FIG. 8 is a partial schematic side view of the web shown in FIG. 7, after folding;

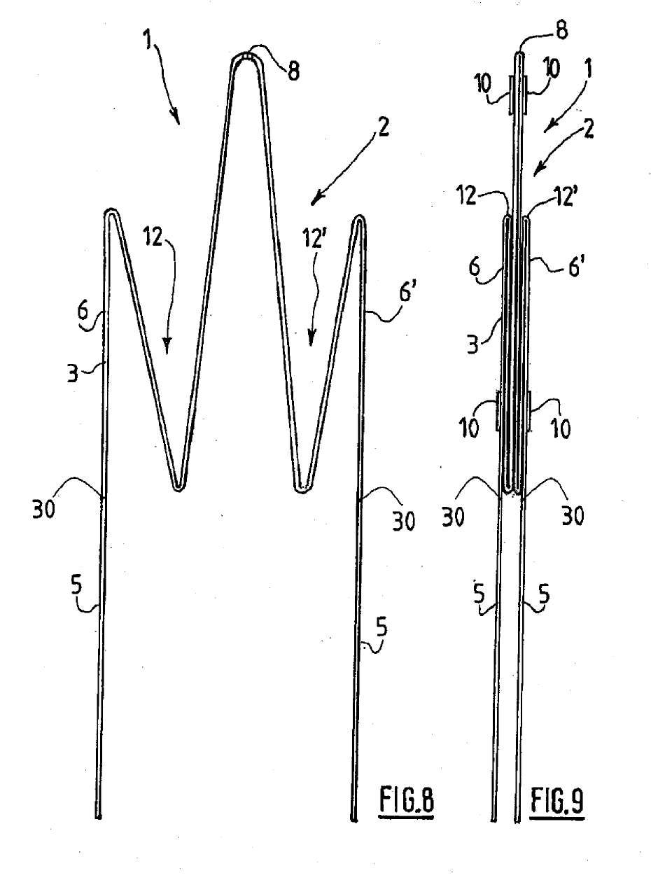

FIG. 9 is a view similar to FIG. 8, once the means of connection by buttoning have been interlocked;

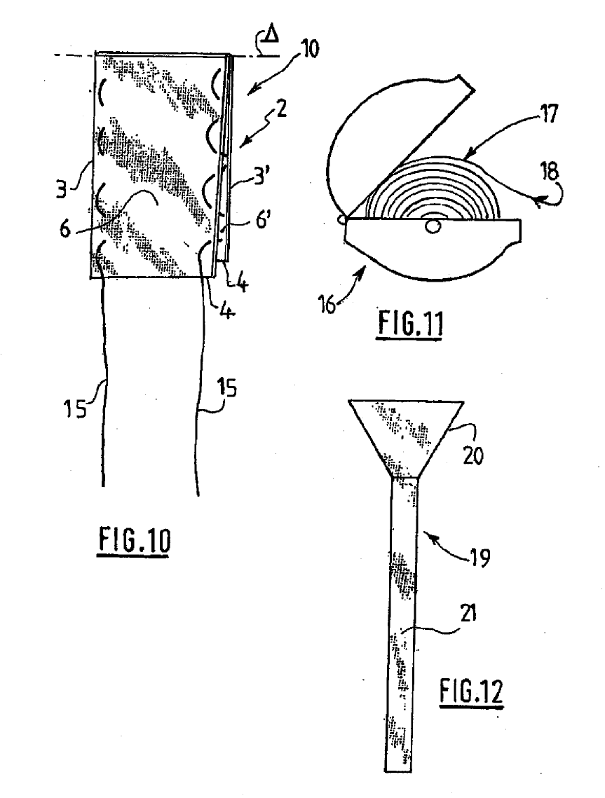

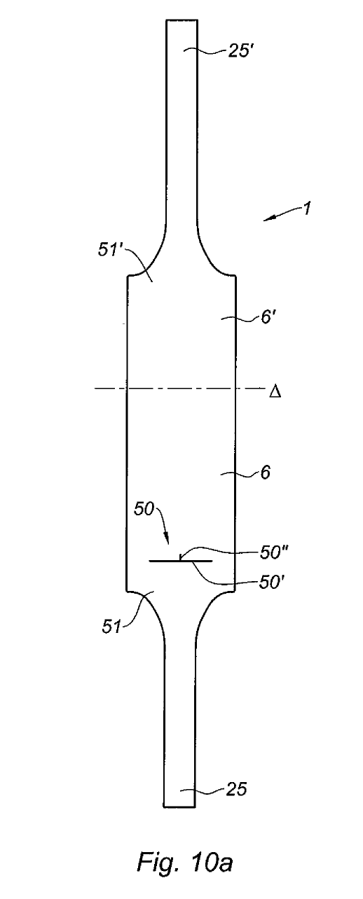

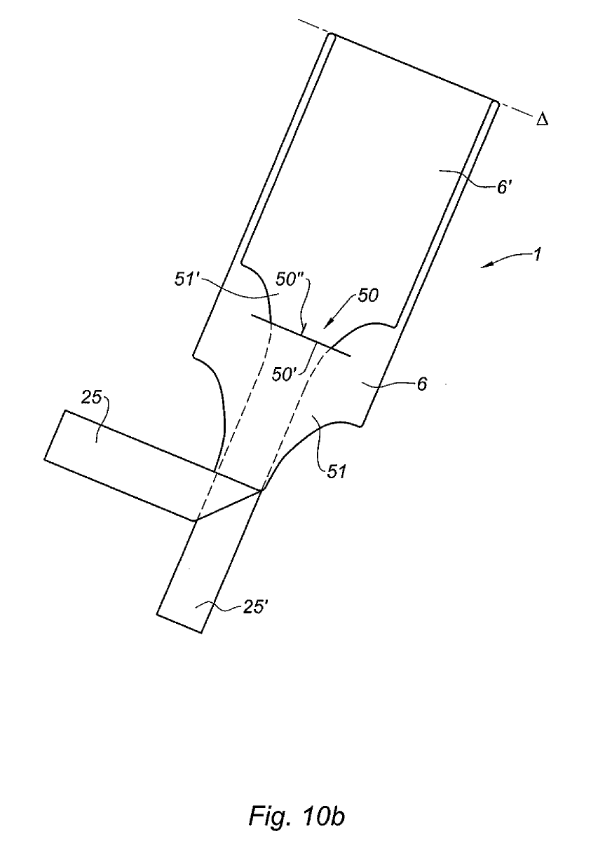

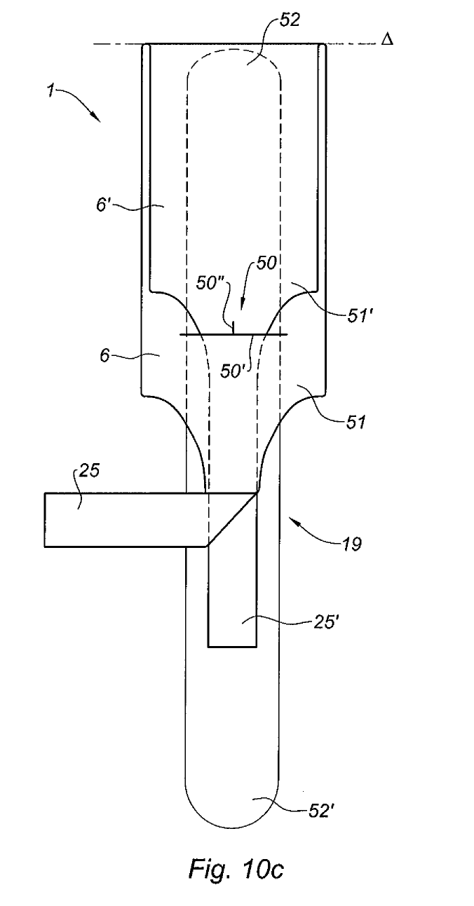

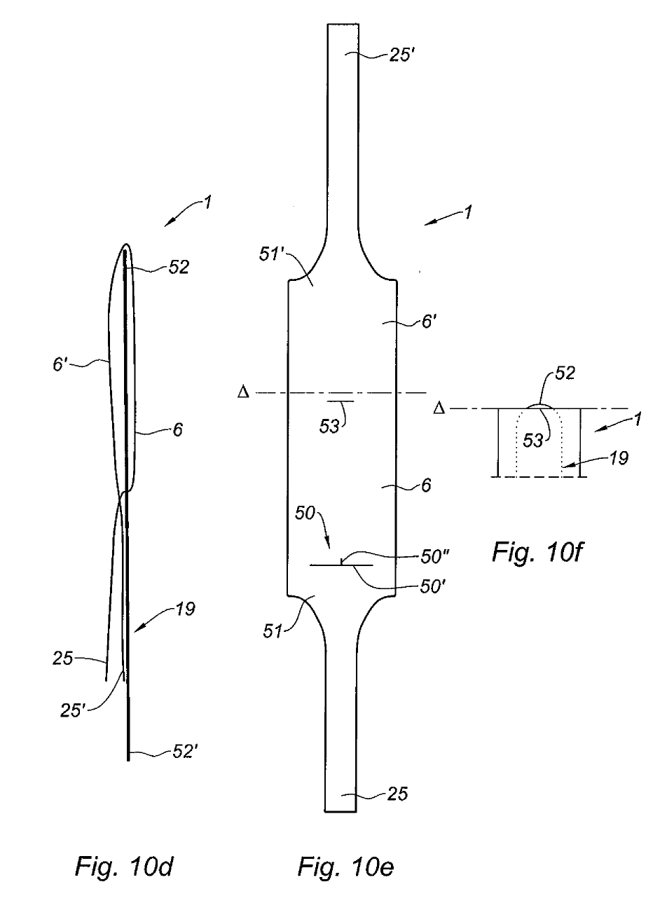

FIG. 10 is a schematic perspective view of another web according to the invention in which the strings constitute means of removal and connecting means;

FIG. 10a is a schematic plan view of a web according to another embodiment of the invention;

FIG. 10b is a view similar to that of FIG. 10a once the web has been folded;

FIG. 10c is a view similar to that of FIG. 10b once a pushing tool has been introduced;

FIG. 10d is a schematic side view of the web shown in FIG. 10c;

FIG. 10e is a schematic plan view of the web shown in FIG. 10a, provided with a supplementary slit;

FIG. 10f is a partial schematic view of the end of a pushing tool engaged in the slit of the web shown in FIG. 10e;

FIG. 11 is a schematic side view of a dispenser according to the invention;

FIG. 12 is a schematic view of a pushing tool with a widened head.

In the following detailed description of the figures defined above, the same elements, or the elements performing identical functions, will keep the same reference signs, so as to make the invention easier to understand.

A web 1 according to the invention, such as is depicted in FIGS. 1 and 2, is made in the form of a rectangular body 2 having two long sides, each constituting a lateral edge 3, 3′, and two short sides 4, each of these being continued by a string 5.

More precisely, the web 1 is integrally formed, that is to say the body 2 and the strings 5 are made in one piece. The material chosen for making the web 1 can, for example, be a non-woven textile of very small thickness.

Once folded about the transverse axis (Δ), the body 2 is divided into two substantially identical flaps 6, 6′ which are superposed on each other, as is shown in FIG. 2.

The length of the web 1 has been chosen in such a way that the length of each flap 6, 6′ is adapted to the depth of the vaginal cavity (not shown) in which the web 1 is intended to be fitted. Moreover, the lengths of the strings 5 are adapted such that they can form means of removal of the web 1 that are accessible from outside the vaginal cavity. In view of the material chosen, these strings 5 do not cause any irritation to the zones through which they pass.

Such a web 1 can be introduced into the vaginal cavity by using a finger which is slipped between the two flaps 6, 6′ or by placing a pushing tool between the two flaps 6, 6′.

In the configuration shown in FIGS. 1 and 2, it will be desirable to use a finger since, as the flaps 6, 6′ are not attached to each other, a web 1 folded in this way has no means of lateral guidance to prevent the head of the pushing tool from disengaging from one of the sides of the web 1 during the insertion of the latter into the vaginal cavity.

The web 1 shown in FIG. 3 differs from the one shown in FIG. 2 in so far as retaining means are provided and are formed by elements 7 that form harpoons, each of them being obtained by a V-shaped slit in the body 2. In view of the orientation of these cuts, the elements 7 forming the harpoons will not offer any resistance during insertion of the web 1 into the vaginal cavity. By contrast, the free tip of each of these elements 7 will tend to anchor itself in the wall of the vagina when the web 1 seeks to migrate outwards from the vaginal cavity.

As is shown in FIG. 4, these retaining means can be supplemented by notches 8 formed in the edge of the body 2. More precisely, each lateral edge 3, 3′ is provided with a plurality of triangular notches 8 arranged in such a way that they are superposed on each other after the web 1 has been folded about the axis Δ.

Preferably, a notch 8 can be formed in each lateral edge 3, 3′ in the area of the axis Δ in order to reduce the cross section of entry of the web 1 in terms of its width and to eliminate any sharp angle. This therefore makes insertion of the web 1 into the vaginal cavity easier and tends to further reduce the risk of irritation.

Moreover, such a web 1 can advantageously incorporate at least one fluid barrier made with the aid of a hydrophobic material, for example petroleum jelly or beeswax. In this case, first and second transverse barriers 30 are arranged in each flap 6, 6′ and in the zone of connection between each string 5 and its corresponding flap 6, 6′. For this purpose, the hydrophobic material is injected into the thickness of the web 1 in order to prevent, or at least slow down, the spread of the fluid along said strings 5. These different fluid barriers 30 will preferably be arranged substantially symmetrically in the web 1 in such a way as to avoid any undesired bonding with other parts of the web 1. It should be noted that reference signs 30 have been indicated only in FIG. 1 so as not to over-complicate the drawing.

The web 1 shown in FIGS. 5 and 6 moreover comprises, on the one hand, means of connection by buttoning and, on the other hand, means for channelling the flows.

First, the means of connection by buttoning are produced by forming slits 9 and buttons 10 in the body 2. More precisely, each lateral edge 3, 3′ of the web 1 comprises two T-shaped buttons 10 obtained by cutting, and also two I-shaped slits 9. These different and complementary connecting means 9, 10 are arranged along the lateral edges 3, 3′ in such a way that, after the web 1 has been folded about the axis (Δ), each of the two buttons 10 is arranged opposite one of the corresponding slits 9. When this has been done, the free head of each button 10 can be forced through the associated slit 9 with the aid of a pushing element or, preferably, a jet of air delivered by a blower (not shown).

Preferably, and as is shown in FIG. 5, two slits 9 and two buttons 10 are formed in the lateral edge 3, respectively at the side of the flap 6 and of the flap 6′, while two buttons 10 and two slits 9 are formed in the lateral edge 3′ respectively at the side of the flap 6 and of the flap 6′.

After assembly, and as is indicated in FIG. 6, the two flaps 6, 6′ are joined to each other by way of the heads of the buttons 10 which have passed through the associated slits 9 and which are turned back so as to be arranged substantially in the same plane as the body 2.

In contrast to an assembly by welding or bonding, for example, this type of connection is particularly advantageous in that it avoids the creation of hard spots that could cause local irritation of the wall of the vagina due to micro-friction.

Moreover, the means for channelling the flow are produced by forming a plurality of slits 11 having an inverted T shape.

When the web 1 is introduced into the vaginal cavity and when the body 2 has deformed, folded or twisted to adapt to the cavity, each of these slits 11 permits the formation of baffles that increase the surface area of contact with the bodily discharges, in all directions. The bodily secretions are often in the form of highly viscous liquids whose rate of discharge is relatively slow. The fact that baffles are formed makes it possible to lengthen the time of contact between the web 1 and the flow and thus to definitively reduce and/or slow down the risk of unwanted discharge thereof along the wall of the vagina.

The web 1 shown in FIG. 7 differs from those described above in that the body 2 has been lengthened in such a way as to permit formation of a pocket 12, 12′ on each flap 6, 6′ by folding.

More precisely, and as has been described above, the body 2 is intended to be folded about the axis Δ in such a way as to be divided into two symmetrical flaps 6, 6′. Moreover, because of the lengthening of the body 2, each of the two flaps 6, 6′ is designed to be able to be folded, on the one hand, about a transverse axis Δ′ and in a direction counter to that corresponding to the folding about the axis Δ, and, on the other hand, about a likewise transverse axis Δ″, in the same direction as that corresponding to the folding about the axis Δ.

Thus, as is shown in FIG. 8, it is possible to form an outwardly oriented pocket 12, 12′ on the flaps 6, 6′, respectively, after the web 1 has been folded about the axes Δ, Δ′ and Δ″.

By forming means of connection by buttoning, as are indicated in FIG. 7, it is then possible, as shown in FIG. 9, to join the two flaps 6, 6′ to each other and at the same time to join each pocket 12, 12′ to the flap 6, 6′ corresponding to it, by way of two buttons 10 designed to pass through all the superposed slits 9 facing them.

This embodiment is particularly advantageous in that, without any hard spot being created, such a web 1 comprises two supplementary zones of retention, making it possible to retain bodily discharges of a volume greater than is anticipated.

Moreover, such a web 1 can advantageously permit the incorporation of at least one fluid barrier 30 formed with the aid of a hydrophobic material, for example petroleum jelly or beeswax. In this case, a fluid barrier 30 is formed approximately in the zone of connection between each string 5 and its corresponding flap 6, 6′. For this purpose, the hydrophobic material is injected into the thickness of the web 1 so as to prevent, or at least slow down, the spread of the fluid along said strings 5.

Lastly, a web 10, such as the one shown in FIG. 10, differs from those described above in that the means of removal are not formed in one piece with the body 2 of the web 10.

In fact, the means of removal are formed by strings 15 that are sewn loosely along the lateral edges 3, 3′ of the body 2 in such a way that the latter is divided into two flaps 6, 6′ which are loosely attached to each other. Each flap 6, 6′ can then execute a relative movement with respect to the other flap 6, 6′. In this way, each flap 6, 6′ will be able to take up the oscillation movements of the vaginal cavity independently of the other flap 6, 6′, the result of which will be to avoid the creation of micro-friction that generates local irritation of the vaginal wall. In this embodiment, it must therefore be understood that the strings 15 act as the means of connecting one flap 6, 6′ to the other and at the same time as means of removal of the web 10.

Another particularly advantageous embodiment of a web 1 according to the invention is shown in FIGS. 10a to 10d.

More precisely, the web 1 comprises two flaps 6, 6′ which can be turned back onto each other by folding them about an axis Δ, as is shown in FIG. 10b. These flaps 6, 6′ are asymmetrical in the sense that the flap 6 is longer than the flap 6′.

The flaps 6, 6′ are continued by strings 25, 25′, respectively. Each of the two strings 25, 25′ has a widened end 51, 51′, respectively, in the zone of connection to its corresponding flap 6, 6′.

The length of the string 25′ is chosen longer than that of the string 15 in such a way as to compensate for the difference in length between the two flaps 6, 6′ and thereby permit easy removal of the web 1.

An opening 50 is formed in proximity to the zone of connection between the flap 6 and the string 25. This opening 50 has substantially the shape of an inverted T and is produced, on the one hand, by making a main cut 50′ parallel to the axis Δ and, on the other hand, by making a central cut 50″ perpendicular to the cut 50′.

Thus, the web 1 can be assembled so as to form a sachet after the free end of the string 25′ has, in a first step, been passed through the opening 50 and then, in a second step, has been pulled until the opposite widened end 51′ comes into abutment against the flap 6.

The widened end 51′ of the web 6′ will thus constitute a point of anchoring in the opening 50, and this local fastening will reduce the risk of downward sliding.

Thus, this embodiment makes it possible to obtain a web 1 forming a sachet in which the traction on the strings 25, 25′ during withdrawal of said web 1 is applied in the centre of the web 1 and not at the periphery, which fact tends to detach the lateral edges of the web 1 from the vaginal cavity and thus facilitates the withdrawal of the web 1 from the latter. At the same time, this makes it possible to further reduce the risks of damage to the wall of the vagina by friction.

The length of the cut 50 will advantageously be chosen so as to be greater than the width of the string 25′, so as to make it easier to pass the string through the opening 50.

The opening 50 does not necessarily have to be arranged at the zone of connection between the string 25 and the flap 6. Indeed, the position of this opening 50 can be left to the choice of the manufacturer, depending on whether or not the latter wishes to give the web 1 a substantial internal volume. This therefore makes it possible to close the two flaps 6, 6, which are arranged towards the bottom once the web 1 has been inserted into the vaginal cavity, and to retain a fluid that will pass inside.

It must be noted that this opening 50 also allows the flow to be channelled towards the inside, which ultimately guarantees a slowing down of the discharge and of leaks.

As is shown in FIGS. 10c and 10d, a pushing tool 19 can then be inserted into the web 1 forming a sachet. More precisely, the pushing tool 19 is introduced into the web 1 via its end 52, which is slid through the opening 50 until it comes into abutment against said web 1 in the area of the axis Δ. Advantageously, the width of the pushing tool 19 (and therefore the length of the cut 50) is quite considerable in order to favour the deployment of the web 1 in the vaginal cavity.

It should be noted that the passage of the string 25′ through the opening 50 can be combined with the passage of the pushing tool 19 through this same opening 50, the pushing tool 19 then acting both as guide and also as abutment.

This embodiment is therefore very advantageous since it makes it possible to obtain a web 1 forming a sachet without the aid of supplementary connecting means, in contrast to the preceding embodiments. Moreover, a single opening 50 is sufficient for the passage of the string 25′ and of the pushing tool 19. Industrial-scale production of the web 1 is thus practical and rapid.

As an alternative, and in order to further facilitate the step of forming the web 1 into a sachet, the end 52′ opposite the end 52 of the pushing tool 19 can be provided with connecting means (not shown), for example a stud, able to cooperate with complementary connecting means (not shown), for example a slit, which are present in the free end of the string 25′. This being so, the free end of the string 25′ is rendered integral with the end 52′ of the pushing tool 19, and it then suffices to slide the end 52 of the latter through the opening 50, then to pull on the pushing tool 19 until the widened end 51′ comes into abutment against the flap 6, such that the web 1 is suitably closed.

It must be understood that the end 52 of the pushing tool 19 inserted into the web can alternatively be in the form of a fork.

Advantageously too, the pushing tool 19 will be chosen to be sufficiently wide in order to optimize the surface area of contact between the web 1 and the vaginal cavity. In this way, it is possible to avoid the web 1 remaining in the shape of a torch once inserted into the vaginal cavity.

As is shown in FIGS. 10e and 10f, the web 1 can also advantageously comprise a slit 53 formed in the flap 6, in proximity to the axis Δ. This slit 53 is substantially parallel to the axis Δ and is sufficiently wide to ensure that part of the end 52 of the pushing tool 19 engages therein. This therefore permits better lateral guiding and support of the pushing tool 19.

Of course, such a web 1 will also be able to be provided with retaining means and/or channelling means such as those described above.

An example of a dispenser 16 according to the invention is given in FIG. 11. This dispenser 16 is of the reel type, and the webs 1, 10 according to the invention are arranged folded in succession between two flexible protective bands 17 wound in a coil.

The bands 17 each comprise a zone of weakness (not shown) arranged between two successive webs 1, 10 in such a way that a user desiring to take out a web 1, 10 simply has to grip the free end 18 of the two bands 17 and exert a shearing force on them such that the bands 17 tear off at the zones of weakness. The user then only has to extract the web 1, 10, sandwiched between the two sections of bands 17 thereby detached, and insert it into the vaginal cavity.

Such a dispenser 16 consequently has several cumulative advantages in so far as it constitutes a package:
which is of very small size,
which is able to contain a large number of webs 1, 10 under very good conditions of storage,
which is environment-friendly since it very substantially reduces the overall amount of packaging.

However, and although this is not the preferred solution, each web 1, 10 can be packaged as a separate unit.

It must be understood that the various webs 1, 10 can each be coated with at least a healing agent and/or lubricating agent and/or antioxidant agent and/or anticoagulant agent, for example, and can be sold with or without pushing tool.

A preferred variant embodiment of a pushing tool consists of a flat, wide rod, such that the surface area of contact between the web 1, 10 and the rod is substantial. By this means it is possible to avoid excessive deformation of the web 1, 10 by twisting, folding, etc., upon its insertion into the vaginal cavity. Consequently, the web 1, 10 is suitably deployed within the cavity, and this maximizes its capacity for retention of the flow that is produced.

In the case where a pushing tool 19 is used, the latter, as shown in FIG. 12, preferably has a widened head 20 forming the end of a grip stem 21 such that optional pockets 12, 12′ formed in the flaps 6, 6′ are masked by the cross section of entry of the web 1, 10 upon insertion of the latter into the vaginal cavity.

Moreover, it must be understood that it may be advantageous, alternatively, to slide the pushing tool between the two flaps 6, 6′ in such a way that it is offcentred, that is to say in such a way that it is positioned nearer one lateral edge 3, 3′ than the other. In fact, it will then be possible to drape the web 1, 10 around the stem of the pushing tool, such that insertion will be made easier because of the reduced width of the cross section of entry of the web 1. This advantage will be all the more pronounced when pockets 12, 12′ have been formed.

A web 1, 10 according to the invention can be produced either by a method of production in which a thickness of appropriate atraumatic material is stamped on a suitable die or in which the same material is cut with the aid of a cutting blade or with the aid of a laser, for example.

In the case of the method of production by stamping, the die will be designed to have elevations and cavities allowing the necessary cuts to be made in the body 2 of the web 1, so as to form retaining means and/or means of connection by buttoning and/or means for channelling the flow.

Although the invention has been described in connection with particular illustrative embodiments, it will be clear that it is not in any way limited to these and that it covers all the technical equivalents of the means described, and their combinations, insofar as these come within the scope of the invention.

1. Flexible retention web designed to be introduced into a vaginal cavity, said web being provided with means of removal and being made of a non-expandable or minimally expandable atraumatic material, the dimensions of which are designed in such a way as to be able to retain and/or slow down bodily discharges of small quantity.

2. Web according to claim 1, wherein it is made of a material chosen from the group comprising a non-woven textile, polyvinyl acetate, cotton, an organic material, a plant material, or a biodegradable material.

3. Web according to claim 1, wherein it has a substantially rectangular, square, oval or circular shape.

4. Web according to claim 1, wherein it is continued by at least two strings constituting the means of removal.

5. Web according to claim 1, wherein the means of removal are formed integrally with said web.

6. Web according to claim 1, wherein the web comprises two flaps, each of them continued by a string constituting a means of removal.

7. Web according to claim 1, further comprising:
two flaps which are folded onto each other and attached to each other by connecting means in such a way as to form two lateral connections.

8. Web according to claim 7, wherein it has two lateral edges, and in that each of the two lateral connections is arranged set back from the lateral edge corresponding to it.

9. Web according to claim 7, wherein the connecting means are formed with the aid of at least two separate, loosely sewn strings.

10. Web according to claim 7, wherein the connecting means are means of connection by buttoning.

11. Web according to claim 10, wherein each flap comprises two lateral edges in each of which the means of connection by buttoning are arranged in the form of at least one slit and/or at least one button, in such a way as to be able to cooperate respectively with at least one complementary button and/or at least one complementary slit of the flap facing it.

12. Web according to claim 11, wherein at least one button has substantially the shape of a T.

13. Web according to claim 6, wherein the first flap is longer than the second flap.

14. Web according to claim 13, wherein the first flap comprises at least one opening for the passage of the string of the second flap after the latter has been turned over onto the first flap by being folded about an axis.

15. Web according to claim 14, wherein the opening is made in the form of a cut substantially shaped as a T or as an inverted T.

16. Web according to claim 13, wherein the string has a widened end at the zone of connection to the second flap.

17. Web according to claim 13, wherein a slit is formed in proximity to the axis and is substantially parallel to the axis.

18. Web according to claim 1, further comprising:
retaining means for limiting the outward migration of said web once it has been introduced into the vaginal cavity.

19. Web according to claim 18, wherein the retaining means comprise at least one element forming a harpoon and obtained from a V-shaped slit in said web.

20. Web according to claim 18, wherein the retaining means comprise at least one notch formed in the edge of the web.

21. Web according to claim 1, further comprising:
means for channelling the bodily discharges.

22. Web according to claim 21, wherein the channelling means are obtained by at least one slit formed in said web.

23. Web according to claim 22, wherein the slit is in the shape of an inverted T.

24. Web according to claim 1, wherein, after folding, it has at least two flaps, each of them provided with at least one pocket in such a way as to increase the capacity for retention of the bodily discharges.

25. Web according to claim 24, wherein each of the pockets is formed by folding a part of the flap on itself and is attached to the latter by connecting means.

26. Web according to claim 25, wherein the connecting means are means of connection by buttoning.

27. Web according to claim 11, wherein the means of connection by buttoning are designed in such a way as to allow the two flaps to be attached to each other while and at the same time to allow each pocket to be attached to its corresponding flap.

28. Web according to claim 1, wherein it incorporates at least one fluid barrier formed with the aid of a hydrophobic material.

29. Method of production of a web according to claim 1, comprising:
stamping a thickness of appropriate atraumatic material on a suitable die.

30. Method of production according to claim 29, further comprising:
cutting out at least one slit and at least one button in the web,
folding the web about an axis, then introducing each button into at least one corresponding slit with the aid of a jet of air or a pushing element.

31. Method of production of a web according to claim 1, comprising:
stamping a thickness of appropriate atraumatic material on a suitable die in such a way as to form a web that is continued at each end by a string.

32. Method of production according to claim 31, further comprising:
cutting out at least one opening in the web,
then passing through the opening the end of the string furthest from said opening, after folding the web onto itself about an axis.

33. Method of production according to claim 32, further comprising:
cutting out at least one slit in the web in proximity to the axis,
inserting an end of a pushing tool through the opening until this end lodges in the slit.

34. Method of production of a web according to claim 1, comprising:
cutting a thickness of appropriate atraumatic material in a predetermined manner with the aid of a cutting tool or a laser.

35. Method of production according to claim 34, further comprising:
cutting out at least one slit and at least one button in the web,
folding the web about an axis, then introducing each button into at least one corresponding slit with the aid of a jet of air or a pushing element.

36. Method of production of a web according to claim 1, comprising:
cutting a thickness of appropriate atraumatic material in a predetermined manner with the aid of a cutting tool or a laser in such a way as to form a web that is continued at each end by a string.

37. Method of production according to claim 36, further comprising:
cutting out at least one opening in the web,
then passing through the opening the end of the string furthest from said opening, after folding the web onto itself about an axis.

38. Method of production according to claim 37, further comprising:
cutting out at least one slit in the web in proximity to the axis,
inserting an end of a pushing tool through the opening until this end lodges in the slit.

39. Dispenser for dispensing a plurality of webs according to claim 1, said dispenser being of the reel type, and the webs being folded in succession between two flexible protective bands.

40. Dispenser according to claim 39, wherein the bands each comprise a zone of weakness between two successive webs.
